(19) United States
(12) Patent Application Publication
(10) Pub. No.: US 2022/0152015 A1
(43) Pub. Date: May 19, 2022

(54) NEDOCROMIL SODIUM TO PREVENT, LIMIT ONSET, AND /OR TREAT PANCREATIC CANCER

(71) Applicant: Charles Owen, Denver, CO (US)

(72) Inventor: Charles Owen, Denver, CO (US)

(21) Appl. No.: 17/528,434

(22) Filed: Nov. 17, 2021

Related U.S. Application Data
(60) Provisional application No. 63/205,105, filed on Nov. 17, 2020.

(51) Int. Cl. A61K 31/4741; A61P 35/00; A61K 9/00; A61K 45/06

(52) U.S. Cl. CPC A61K 31/4741; A61P 35/00; A61K 45/06; A61K 9/0019; A61K 9/0021

(57) ABSTRACT

Provided herein are compositions, including Nedocromil Sodium, and methods to prevent, limit onset, and/or treat Pancreatic Cancer in a subject. Nedocromil sodium can be combined with chemotherapy and other medicaments to prevent, limit onset, and or treat Multi drug resistance Pancreatic Cancer

### RELATED APPLICATIONS

This application claims priority to U.S. Provisional Application Ser. No. 63/205,105 filed Nov. 17, 2020 and entitled “Nedocromil Sodium to prevent, limit onset, and/or treat multi drug resistance pancreatic cancer, which is hereby incorporated by reference in its entirety.

### TECHNICAL FIELD

The present disclosure relates to the prevention, limitation and/or treatment of Pancreatic Cancer, and in particular, using Nedocromil Sodium and its analogs to facilitate the prevention, limitation of onset and/or treatment of Pancreatic Cancer.

### BACKGROUND

The statements in this section merely provide background information related to the present disclosure and may not constitute prior art.

Pancreatic cancer arises when cells in the pancreas, a glandular organ behind the stomach, begin to multiply out of control and form a mass. These cancerous cells have the ability to invade other parts of the body. The most common, pancreatic adenocarcinoma, accounts for about 90% of cases, and the term “pancreatic cancer” is used to refer to that type. These adenocarcinomas start within the part of the pancreas that makes digestive enzymes. Pancreatic cancer has the worst survival rate of all cancers. The current standard care for metastatic pancreatic cancer is gemcitabine, however, the success of this treatment is poor and overall survival has not improved for decades. Drug resistance (both intrinsic and acquired) is thought to be a major reason for the limited benefit of most pancreatic cancer therapies.

One of the important features of pancreatic cancer is the high resistance to traditional chemo- and radiation therapy, including both intrinsic (de novo) and acquired (therapy-induced) chemoresistant behaviors of the cancer cells. Patients with pancreatic cancer usually present with locally advanced, unresectable or metastatic diseases. Even for patients with resectable disease, most of them will relapse. Therefore, postoperative chemo- or radiation therapy is still necessary for pancreatic cancer management although there is argument that preoperative therapy (neoadjuvant treatment) works better than the postoperative therapy, and other people think chemo- and radiation therapy is not necessary at all.
Aberrant gene expression
Mutation
Deregulation of key signaling pathways (such as NF-κB, Akt, and apoptosis pathways),
Epithelial-mesenchymal transition (EMT) and the presence of stroma cells
Efflux mechanisms to remove chemotherapy
Highly resistant cells and stem cells

Pancreatic cancer is one of the most difficult human cancers to treat due to the inability to detect disease at an early stage and the lack of effective therapies. Although there has been some progress in the use of improved diagnostic methods and development of novel targeted therapies, the overall survival rate has not improved over the last decade. Pancreatic cancer remains the fourth leading cause of cancer death in the United States, with an annual mortality nearly equivalent to the annual incidence. Eighty percent of pancreatic adenocarcinomas are unresectable in patients with clinical symptoms. Clearly, there is a need to understand more about the molecular mechanisms of pancreatic cancer pathogenesis and to develop effective treatments for pancreatic cancer.

Conventional treatment of Pancreatic Cancer varies depending on the perceived underlying cause. Most Pancreatic Cancer have to be treated with chemotherapy with selection based on the type of Pancreatic Cancer involved. The traditional route of administration is by IV injectable, intravenous fluid therapy and oral. Anti-inflammatory drugs are used to decrease inflammation. The most commonly used chemotherapy for pancreatic cancer, gemcitabine, has modest clinical benefit and may not improve overall survival to a clinically meaningful degree. The lack of significant clinical response of pancreatic cancer patients to chemotherapy is likely due to the inherent chemo-resistance of pancreatic cancer cells as well as impaired drug delivery pathways. Understanding the underlying mechanisms of drug resistance in pancreatic cancer is critical to develop new effective treatments for this deadly disease.

Impaired drug delivery pathways have been shown to be another reason for the drug resistance in pancreatic cancer. It is believed that within pancreatic tumors there are solid tumor masses which lack adequate vasculature with intensive desmoplastic reaction. Therefore, it is difficult for any drug to penetrate the stroma area and reach the inside of the solid tumors. It is also suggested that chemotherapy can further activate the drug resistant pathway in pancreatic cancer.

The present invention is directed toward overcoming one or more of the problems discussed above, including novel compositions and administration methods for preventing, limiting the onset or treating multidrug resistance Pancreatic Cancer.

Nedocromil Sodium-Nedocromil Sodium is a pyranoquinoline with the chemical name 4H-Pyrano[3,2-g]quinoline-2,8-dicarboxylic acid, 9-ethyl-6,9-dihydro-4,6-dioxo-10-propyl-, disodium salt, and it has a molecular weight of 415.3. The empirical formula is C19H15NNa2O7. Nedocromil Sodium, a yellow powder, is soluble in water.

Against this backdrop the present disclosure is provided.

### SUMMARY

Provided herein are innovations with respect to preventing, limiting onset and treating Pancreatic Cancer.

In one embodiment, a method is provided for the prevention of Pancreatic Cancer. The method comprises identifying a subject at risk of developing Pancreatic Cancer and administering Nedocromil Sodium by Intra Arterial or other delivery routes which may include oral, intravenous injection, intramuscular injection, subcutaneous injection, inhaled, buccal, intranasal, subdermal, transdermal and intravenous fluid therapy.

In another embodiment, a method is provided for limiting onset of Pancreatic Cancer. The method comprises identifying a subject in early onset of Pancreatic Cancer and administering Nedocromil Sodium by Intra Arterial or other delivery routes which may include oral, intravenous injection, intramuscular injection, subcutaneous injection, inhaled, buccal, intranasal, subdermal, transdermal and intravenous fluid therapy.

In yet another embodiment, a method is provided for treating Pancreatic Cancer. The method comprises identifying a subject having Pancreatic Cancer and administering Nedocromil Sodium, by Intra-Arterial or other delivery routes which may include oral, intravenous injection, intramuscular injection, subcutaneous injection, inhaled, buccal, intranasal, subdermal, transdermal and intravenous fluid therapy.

In embodiments described here in Nedocromil Sodium—Nedocromil Sodium is a pyranoquinoline with the chemical name 4H-Pyrano[3,2-g]quinoline-2,8-dicarboxylic acid, 9-ethyl-6,9-dihydro-4,6-dioxo-10-propyl-, disodium salt, and it has a molecular weight of 415.3. The empirical formula is C19H15NNa2O7. Nedocromil Sodium, a yellow powder, is soluble in water. Route of administration can be performed once or multiple times over the course of one or more weeks.

These and other feature as well as advantages which characterize the invention will be apparent from reading of the following detailed description and a review of appended claims.

### DETAILED DESCRIPTION

The following description is merely exemplary in nature and is in no way intended to limit the present disclosure, application, or uses.

Provided herein are methods and compositions for preventing, limiting onset and/or treating Pancreatic Cancer. These methods include preventing, limiting onset, and/or treating (including mitigating the severity of symptoms) acute Pancreatic Cancer, subclinical Pancreatic Cancer and chronic Pancreatic Cancer. The compositions comprise of, or consist of, or consist essentially of Nedocromil Sodium, 5% concentration or more of Nedocromil Sodium, analogs of Nedocromil Sodium, 5% concentration or more of Nedocromil Sodium, alone or in combination with a further medicament or carrier. The methods comprise, consist of, or consist essentially of administration of Nedocromil Sodium, 5% concentration or more of Nedocromil Sodium, analogs of Nedocromil Sodium, alone or in combination with a further medicament to a subject to prevent, limit onset and/or treat multi drug resistance Pancreatic Cancer. The methods can include administering Nedocormil Sodium, 5% concentration or more of Nedocromil Sodium, analogs of Nedocromil Sodium, alone or in combination by Intra arterial, microneedle transdermal patch or other delivery routes which may include oral, intravenous injection, intramuscular injection, subcutaneous injection, inhaled, buccal, intranasal, subdermal, transdermal and intravenous fluid therapy. In some aspects of the method other medicaments are co-administered to the subject in conventional manners, while Nedocromil Sodium, 5% concentration or more of Nedocromil Sodium, analogs of Nedocromil Sodium is administered via Intra Arterial.

Pancreatic Cancer

Pancreatic cancer begins in the tissues of your pancreas-an organ in your abdomen that lies behind the lower part of your stomach. Your pancreas releases enzymes that aid digestion and produces hormones that help manage your blood sugar. Several types of growths can occur in the pancreas, including cancerous and noncancerous tumors. The most common type of cancer that forms in the pancreas begins in the cells that line the ducts that carry digestive enzymes out of the pancreas (pancreatic ductal adenocarcinoma). Pancreatic cancer is seldom detected at its early stages when it's most curable. This is because it often doesn't cause symptoms until after it has spread to other organs.

Pancreatic cancer treatment options are chosen based on the extent of the cancer. Options may include surgery, chemotherapy, radiation therapy or a combination of these, but have only a 7% success rate.

Through the discussion throughout is generally directed toward subjects with Pancreatic Cancer, methods and compositions are equally applicable to any type of Cancer herein can include, but is not limited to:
Bladder Cancer
Breast Cancer
Colon and Rectal Cancer
Endometrial Cancer
Kidney Cancer
Leukemia
Liver Cancer
Lung Cancer
Melanoma
Non-Hodgkin Lymphoma
Prostate Cancer
Thyroid Cancer.

Cell Mediated Immune Stress Response Developmental Phase

In general, Pancreatic Cancer is initiated by the cell mediated immune stress response. Cell mediated immunity does not involve antibodies, but rather involves the activation of phagocytes, antigen-specific cytotoxic T-lymphocytes, and the release of various cytokines in response to an antigen.

Pancreatic Cancer and its components are the antigen in a cell mediated immune response: Cellular malfunction resulting from homeostatic imbalance is believed to be an underlying factor responsible for most Pancreatic Cancer infection and antimicrobial resistance. The point of imbalance is any location where the body tissue is compromised such as wounds, cancerous organ, lung, mouth, GI tract etc. Antimicrobial resistance Pancreatic Cancer are Opportunistic facultative anaerobic Pancreatic Cancer where host defense mechanisms have been compromised and cellular malfunction is taken place.

While not wishing to be bound by theory, it is believed that the first major pathway which is activated during the point of imbalance is the immune response pathway which results in the activation of the autonomic nervous system, and the secretion of catecholamines. (epinephrine).

Epinephrine

Epinephrine (endogenous ligand) is a neurotransmitter normally produced by both the adrenal glands and certain neurons. It plays an important role in the flight or fight response by shunting blood flow to muscles and vaso-constriction of the blood vessels resulting in hypoxia within the host cell and having selective and high affinity binding to G protein-coupled receptor 35 in the cytoplasmic membrane of facultative anaerobic resistance Pancreatic Cancer from the upset of homeostasis and the cell mediated immune response.

G Protein Coupled Receptor 35

G protein coupled receptors (GPCR), constitute a large protein family of receptors that detect molecules outside the cell and activate internal signal transduction pathways and, ultimately, cellular responses. Evidence suggests that receptors and G proteins are actually pre-coupled.

G protein coupled receptor 35 is a G protein-coupled receptor which in humans is encoded by the GPR35 trans-membrane gene of the select cell immune response pathway. Heightened expression of GPR35 is found in all immune cells.

G protein 35 biological process consist of, but not limited to:
Positive regulation of cytosolic calcium ion concentration
Positive regulation of Rho protein signal transduction
Chemokine-mediated signaling pathway
Cytoskeleton organization=DNA
Negative regulation of voltage-gated calcium channel activity
Negative regulation of neuronal action potential
G-protein coupled receptor signaling pathway
GPR35 interacts with CXCL17 in breast cancer cells

G Protein Gated Ion Channel

G protein-gated ion channels are specific ion channels located in the plasma membrane of cells that are directly activated by G protein couple receptor 35. The binding of epinephrine (endogenous ligand) to G protein couple receptor 35 that is encased in the cytoplasmic membrane of facultative anaerobic resistance Pancreatic Cancer creates a conformational change towards a permanently active receptor state and opens the G protein gated ion channel that is component of G protein couple receptor 35 within the membrane and dramatically increases extracellular ca2+ for transport.

Calcium Ion

Calcium ions (Ca²⁺) play a vital role in the physiology and biochemistry of organisms and most important of the facultative anaerobic resistance Pancreatic Cancer cell. Calcium ions, like many other ions, are of such vital importance to many physiological processes that its concentration is maintained within specific limits to ensure adequate homeostasis. It has been found that calcium ions are involved in Pancreatic Cancer maintenance of cell structure, motility, transport and cell differentiation.

Calcium Ion (ca2+) is the mediator Pancreatic Cancer. Calcium ion influx (ca2+) is the mechanism by which microorganism exhibit resistance to chemotherapy by decreasing drug permeability and increasing active efflux (pumping out) of the chemotherapy drugs across the cell surface by the calcium ion efflux pump (Pancreatic Cancer efflux pump) powered by ATP. Without intracellular ca2+ influx Pancreatic Cancer cannot continue to invade host cells and continue to activate infection and antimicrobial resistance.

The resting concentration of Ca2+ in the cytoplasm is normally maintained around 100 nM, variously reported as 20,000- to 100,000-fold lower than typical extracellular concentration. The opening of the G protein gated channel triggers a sudden increase in the cytoplasmic Ca2+ level up to 500-1,000 nM triggering a toxic situation within the cell and initiates the Pancreatic Cancer efflux pump powered by ATP to actively pump the Ca2+, and chemotherapy back out of the cytoplasm and return the cell to its pre-signal state to maintain homeostasis.

Pancreatic Cancer Efflux Pump

Pancreatic Cancer efflux pumps comprise a tripartite structure, consisting of an efflux transporter with broad-substrate specificity, (ca2+) an outer membrane channel that carries substrates from the pump through the outer membrane (G protein gated ion channel) and a periplasmic adapter protein which is G Protein coupled receptor 35. The G protein coupled receptor 35 is required for stable association of the inner and outer membrane components of the pump.

As stated, a toxic situation within the cell and initiates the Pancreatic Cancer efflux pump powered by ATP to actively pump the Ca2+, Pancreatic Cancer proteins and chemotherapy back out of the cytoplasm.

Compositions and methods described herein are useful in preventing pancreatic cancer in this stage. For example, if a physician, medical doctor, specialist, personal caregiver, nurse, etc., identifies a subject that may be at risk of pancreatic cancer as the subject exhibits a change in behavior conductive with multi drug resistance pancreatic cancer, because the subject exhibits physical signs of pancreatic cancer, or because a subject was exposed to a cell mediated immune response with the potential to initiate pancreatic cancer. Nedocromil Sodium, analogs of Nedocromil Sodium, 5% concentration or more of Nedocromil Sodium alone or in combination with a further medicament or carrier can be administered. The methods comprise, consist of, or consist essentially of administration of Nedocromil Sodium, analogs of Nedocromil Sodium, 5% concentration or more of Nedocromil Sodium, alone or in combination with a further medicament to a subject to prevent, limit onset and/or treat Pancreatic Cancer. The methods can include administering Nedocromil Sodium, analogs of Nedocromil Sodium, 5% concentration or more of Nedocromil Sodium alone or in combination by Intra Arterial administration or other delivery routes which may include oral, intravenous injection, intramuscular injection, subcutaneous injection, inhaled, buccal, intranasal, subdermal, transdermal and intravenous fluid therapy. In some aspects of the method other medicaments are co-administered to the subject in conventional manners, while Nedocromil Sodium, analogs of Nedocromil Sodium, 5% concentration or more of Nedoocromil Sodium, is administered via Intra-arterial administration.

Acute Phase

The binding of epinephrine (endogenous ligand) to G protein couple receptor 35 that is encased in the cytoplasmic membrane of facultative anaerobic resistance Pancreatic Cancer creates a conformational change towards a permanently active receptor state and opens the G protein gated ion channel that is a component of G protein couple receptor 35 within the membrane and dramatically increases extracellular ca2+ for transport into the Pancreatic Cancer cell to cause apoptosis. To maintain cell homeostasis and cell survival, the Pancreatic Cancer efflux pump is activated to pump ca2+ and chemotherapy out of the cell along with Pancreatic Cancer proteins that assist multi drug resistance Pancreatic Cancer invasion of the host cell. In a cell mediated immune response the host cell is macrophages.

Macrophages

Macrophages are a type of white blood cell, of the immune system, that engulfs and digests Pancreatic Cancer that does not have the type of proteins specific to healthy body cells on its surface in a process called phagocytosis. These large phagocytes are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in nonspecific defense (innate immunity) and also help initiate specific defense mechanisms (adaptive immunity) by recruiting other immune cells such as lymphocytes. For example, they are important as antigen presenters to T cells. In humans,

In a cell mediated immune response, epinephrine also binds the g protein coupled receptor 35 of the macrophage initiating hypoxia along with creating a conformational change towards a permanently active receptor state and opens the G protein gated ion channel that is component of G protein couple receptor 35 within the macrophage cell allowing for facultative anaerobic resistance Pancreatic Cancer to invade the cell along with ca2+ influx.

As soon as a Pancreatic Cancer pathogen enters the macrophage host cell, the host tries to degrade the Pancreatic Cancer in the lysosome; thus, Pancreatic Cancer pathogens need to prevent, delay, or escape contact with lysosomes Hypoxia reprograms human macrophages towards a pro inflammatory direction and disables the lysosomes mechanism to the invading Pancreatic Cancer allowing for colonization.

At this point, it is said that the facultative anaerobic resistance Pancreatic Cancer has invaded and hijacked the host cell by binding to ca2+ activating Pancreatic Cancer toxin proteins to multiply as the Pancreatic Cancer grows within the hypoxia macrophage host cell.

The influx of ca2+ has raised the calcium level within the cell causing a toxic effect. The resting concentration of Ca2+ in the cytoplasm is normally maintained around 100 nM, variously reported as 20,000- to 100,000-fold lower than typical extracellular concentration. The opening of the G protein gated channel triggers a sudden increase in the cytoplasmic Ca2+ level up to 500-1,000 nM triggering a toxic situation within the cell and initiates the Pancreatic Cancer efflux pump powered by ATP to actively pump the Ca2+ and Pancreatic Cancer toxins back out of the cell and return the cell to its pre-signal state to maintain homeostasis and to maintain Pancreatic Cancer survival within the host cell.

Histamine

Pancreatic Cancer are capable of producing histamine using histidine decarboxylase enzymes unrelated to those found in animals where it functions as a neurotransmitter.

Antigen Presenting Cells

An antigen-presenting cell (APC) or accessory cell is a cell that displays antigen complexed with major histocompatibility complexes (MHCs) on their surfaces; this process is known as antigen presentation. Almost all cell types can serve as some form of APC and are found in a variety of tissue including macrophage. The presenting antigen originating inside the Pancreatic Cancer invaded macrophage cell is histamine acting as a neurotransmitter and attaching itself to the surface of the macrophage for recognition.

Helper T Cells

The T helper cells (Tₕ cells), also known as CD4 cells, are a type of T cell that play an important role in the immune system, particularly in the adaptive immune system. Mature Tₕ cells express the surface protein CD4 and are referred to as CD4⁺ T cells. Such CD4⁺ T cells are generally treated as having a pre-defined role as helper T cells within the immune system. For example, when an antigen-presenting cell expresses an antigen, such as histamine in the cell mediated immune response, on MHC class II, a CD4⁺ cell will aid those cells through a combination of cell to cell with Histamine having selective and high affinity binding to G coupled receptor 35 (H 4 receptor) located on the mast cell.

Mast Cell

Mast Cells reside in several types of tissues and in the cell mediated immune response resides in mucosal tissue. Mast cells contain histamine as a granule different from histamine as a neurotransmitter. Mast cells also contain metalloproteinase-2 and -9 as well as dopamine and serotonin. Mast cells are involved in the inflammatory process, rapidly releasing granules into the mucosal tissue upon activation by ca2+.

G Protein Coupled Receptor 35

G protein coupled receptors (GPCR), constitute a large protein family of receptors that detect molecules outside the cell and activate internal signal transduction pathways and, ultimately, cellular responses. Evidence suggests that receptors and G proteins are actually pre-coupled. Heightened expression of GPR35 is found in the mast cell.

G Protein Gated Ion Channel

G protein-gated ion channels are specific ion channels located in the plasma membrane of cells that are directly activated by G protein couple receptor 35.

The binding of Histamine (endogenous ligand) to G protein couple receptor 35 that is encased in the cytoplasmic membrane of the mast cell creates a conformational change towards a permanently active receptor state and opens the G protein gated ion channel that is component of G protein couple receptor 35 within the membrane and dramatically increases extracellular ca2+ for transport.

Calcium Ion

Calcium ions (Ca²⁺) play a vital role in the physiology and biochemistry of the mast cell. Calcium ions, like many other ions, are of such vital importance to many physiological processes that its concentration is maintained within specific limits to ensure adequate homeostasis. It has been found that calcium ions are involved in mast cell degranulation.

The resting concentration of Ca2+ in the cytoplasm is normally maintained around 100 nM, variously reported as 20,000- to 100,000-fold lower than typical extracellular concentration. The opening of the G protein gated channel triggers a sudden increase in the cytoplasmic Ca2+ level up to 500-1,000 nM triggering a toxic situation within the cell and initiates the mast cell efflux pump powered by ATP to actively pump the Ca2+, and mediator granules back out of the cytoplasm and return the cell to its pre-signal state to maintain homeostasis.

Mast Cell Efflux Pump

Mast cell efflux pumps comprise a tripartite structure, consisting of an efflux transporter with broad-substrate specificity, (ca2+) an outer membrane channel that carries substrates from the pump through the outer membrane (G protein gated ion channel) and a periplasmic adapter protein which is G Protein coupled receptor 35. The G protein coupled receptor 35 is required for stable association of the inner and outer membrane components of the pump.

Mast Cell Degranulation

A unique, stimulus-specific set of mast cell mediators is released through degranulation following the activation of G protein coupled receptor 35 on mast cells.^{[10]} Examples of mediators that are released into the extracellular environment during mast cell degranulation include:
serine proteases, such as tryptase and chymase
histamine (2-5 picograms per mast cell)
serotonin
proteoglycans, mainly heparin (active as anticoagulant) and some chondroitin sulfate proteoglycans
adenosine triphosphate (ATP)
lysosomal enzymes
   β-hexosaminidase
   β-glucuronidase
   arylsulfatases
newly formed lipid mediators (eicosanoids):
   thromboxane
   prostaglandin D2
   leukotriene C4
   platelet-activating factor
cytokines
   TNF-α
   basic fibroblast growth factor
   interleukin-4
   stem cell factor
   chemokines, such as eosinophil chemotactic factor
reactive oxygen species

Acute Inflammation

Mast cell degranulation of inflammatory mediators listed above initiate acute inflammation in the mucosal tissue. Local swelling, redness, heat and pain are characteristics of acute inflammation. As acute inflammation escalates, vascular permeability increase, causing Pancreatic Cancer infection and the spread of Pancreatic Cancer infection within the mucosal tissue.

Compositions and methods described herein are useful in limiting onset of multidrug resistance Pancreatic Cancer in this stage. For example, if a physician, medical doctor, specialist, personal caregiver, nurse, etc., identifies a subject that may be at onset of Pancreatic Cancer as the subject exhibits a change in behavior conductive with multi drug resistance Pancreatic Cancer, because the subject exhibits physical signs of Pancreatic Cancer, or because a subject was exposed to a cell mediated immune response with the potential to initiate Pancreatic Cancer. Nedocromil Sodium, analogs of Nedocromil Sodium, 5% concentration or more of Nedocromil Sodium alone or in combination with a further medicament or carrier can be administered. The methods comprise, consist of, or consist essentially of administration of Nedocromil Sodium, analogs of Nedocromil Sodium, 5% concentration or more of Nedocromil Sodium alone or in combination with a further medicament to a subject to prevent, limit onset and/or treat Pancreatic Cancer.

The methods can include administering Nedocromil Sodium, analogs of Nedocromil Sodium, 5% concentration or more of Nedocromil Sodium alone or in combination by Intra Arterial administration or other delivery routes which may include oral, intravenous injection, intramuscular injection, subcutaneous injection, inhaled, buccal, intranasal, subdermal, transdermal, and intravenous fluid therapy.

In some aspects of the method other medications are co-administered to the subject in conventional manners, while Nedocromil Sodium, analogs of Nedocromil Sodium, 5% concentration or more of Nedocromil Sodium is administered via Intra-arterial.

Chronic Phase

In circumstances, when resolution of cellular imbalance cannot be achieved, the mucosal tissue will suffer from repeated invasion of Pancreatic Cancer and acute inflammation characterized by continuous Pancreatic Cancer infection. This becomes a vicious cycle with metastasis development and tissue destruction in the subject causing severe long term illness or even death.

Compositions and methods described herein are useful in treating Pancreatic Cancer in this stage. For example, if a physician, medical doctor, specialist, personal caregiver, nurse, etc., identifies a subject that is in need of treatment of Pancreatic Cancer as the subject exhibits a change in behavior conductive with Pancreatic Cancer, because the subject exhibits physical signs of Pancreatic Cancer, or because a subject was exposed to a cell mediated immune response with the potential to initiate Pancreatic Cancer. Nedocromil Sodium, analogs of Nedocromil Sodium, 5% concentration or more of Nedocromil Sodium alone or in combination with a further medicament or carrier can be administered. The methods comprise, consist of, or consist essentially of administration of Nedocromil Sodium, analogs of Nedocromil Sodium, 5% concentration or more of Nedocromil Sodium, alone or in combination with a further medicament to a subject to prevent, limit onset and/or treat Pancreatic Cancer.

The methods can include administering Nedocromil Sodium, analogs of Nedocromil Sodium, 5% concentration or more of Nedocromil Sodium alone or in combination by Intra arterial administration other delivery routes which may include oral, intravenous injection, intramuscular injection, subcutaneous injection, inhaled, buccal, intranasal, subdermal, transdermal and intravenous fluid therapy. In some aspects of the method other medicaments are co-administered to the subject in conventional manners, while Nedocromil Sodium, analogs of Nedocromil Sodium, 5% concentration or more of Nedocromil Sodium. Nedocromil Sodium, is administered via intra-arterial.

Compositions

The compositions herein can be used for preventing, limiting onset, and/or treating drug Pancreatic Cancer. Note that treating multi drug resistance Pancreatic Cancer refers to mitigating the severity of Pancreatic Cancer, including reduction in any one symptom of Pancreatic Cancer. In some cases, treating Pancreatic Cancer results in complete removal of the symptoms of Pancreatic Cancer as determined by health care specialist.

In some embodiments, the compositions herein are used to treat Pancreatic Cancer.

In still other embodiments, the compositions herein are used to prevent Pancreatic Cancer.

In still other embodiments, the compositions herein are used to limit the onset of Pancreatic Cancer.

Thus, in some cases, a composition herein is administered to a subject to treat, mitigate the severity of, limit onset of, or prevent Pancreatic Cancer and in particular, to treat, mitigate the severity of, limit the onset of, or prevent acute Pancreatic Cancer, subclinical Pancreatic Cancer and/or chronic Pancreatic Cancer. These compositions can be administered one time or multiple times over the course of one day, several days or several months

Nedocromil Sodium

Nedocromil Sodium is a pyranoquinoline with the chemical name 4H-Pyrano[3,2-g]quinoline-2,8-dicarboxylic acid, 9-ethyl-6,9-dihydro-4,6-dioxo-10-propyl-, disodium salt, and it has a molecular weight of 415.3. The empirical formula is C19H15NNa2O7. Nedocromil Sodium, a yellow powder, is soluble in water.

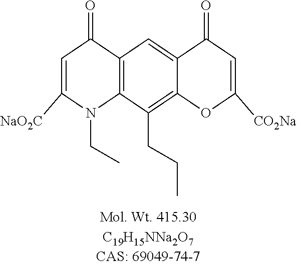

Nedocromil Sodium has high affinity bind or “ligand bias” and is a very potent full agonist to GPCR 35 and has the capability to displace the binding effectiveness of epinephrine to GPCR 35 and signals GPCR to close the G protein gaited ion channel. The closure represents the decrease of calcium ion influx and efflux activating apoptosis of the Pancreatic Cancer cell in Pancreatic Cancer. Nedocromil sodium is not only an inhibitor of the immunological release of inflammatory mediators, but also extends this activity to mucosal mast cells, which are thought to play an important role in Pancreatic ductal adenocarcinoma (PDAC). The conclusion that nedocromil sodium is an anti-inflammatory agent is supported by in vivo observations of its capacity to inhibit the late response to antigen challenge is commonly considered a mast cell stabilizer based on its ability to prevent secretion from mast cells. An attractive model for the actions of Nedocromil Sodium on mast cells is based upon its ability to interact with a component of a regulated Ca2+.

Nedocromil Sodium, analogs of Nedocromil Sodium can be used to prevent, limit the onset, and/or treat acute Pancreatic Cancer, subclinical multi drug resistance Pancreatic Cancer and chronic Pancreatic Cancer. In some embodiments, a combination of two or more compounds described herein e.g. a combination of 50% Nedocromil Sodium and 50% Nedocromil Sodium or 50% Nedocromil Sodium and an analog of 50%.

Nedocromil Sodium, analogs of (Nedocromil Sodium) can be used in combination with other medications used to treat the underlying cause of Pancreatic Cancer or symptoms associated with multi drug resistance Pancreatic Cancer. These medications can be administered in the same formulation or in separate formulations, by the same route or by different routes, and at the same time or at different times.

Typical medications useful in combination with, analogs of (Nedocromil Sodium) include chemotherapy. Exemplary chemotherapy include:
Abraxane (Paclitaxel Albumin-stabilized Nanoparticle Formulation)
Afinitor (Everolimus)
Erlotinib Hydrochloride
Everolimus
5-FU (Fluorouracil Injection)
Fluorouracil Injection
Gemcitabine Hydrochloride
Gemzar (Gemcitabine Hydrochloride)
Irinotecan Hydrochloride Liposome
Lynparza (Olaparib)
Mitomycin C
Olaparib
Onivyde (Irinotecan Hydrochloride Liposome)
Paclitaxel Albumin-stabilized Nanoparticle Formulation
Tarceva (Erlotinib Hydrochloride)

Drug Combinations Used in Pancreatic Cancer:
FOLFIRINOX
GEMCITABINE-CISPLATIN
GEMCITABINE-OXALIPLATIN

When administered in combination with Nedocromil Sodium, analogs of, (Nedocromil Sodium) chemotherapy can be used to inhibit activity of any secondary Pancreatic Cancer release regardless of the presence of Nedocromil Sodium, analogs of Nedocromil Sodium).

Formulations

The compositions herein can be formulated with one or more carriers or excipients for delivery to a subject. Such carriers can be for example, pharmaceutical carriers and veterinary carriers.

Typically, such formulations will include one or more acceptable carriers, excipients, or diluents. Pharmaceutically acceptable carriers for therapeutic use are well known in the pharmaceutical art, and are described, e.g., in Remington's Pharmaceutical Sciences, Gennaro, A R ed., 20^{th} edition, 2000; Williams and Wilkins PA, USA, which is incorporated herein by reference for all purposes. Veterinary excipients and carriers are also known in the art.

A pharmaceutical formulation can also contain any kind of other compatible ingredients such as, for example, protective colloids, adhesives, thickening agents, thixotropic, agents, penetrating agents, stabilizing agents, sequestering agents, fertilizers, anti-freeze agents, Repellents, color additives, corrosion inhibitors, water repelling agents, UV stabilizers, pigments, dyes or polymers.

In some embodiments, the compositions herein may be formulated as a salt and be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preparations may be lyophilized powder, which is combined with buffer prior to use.

After pharmaceutically and physiologically acceptable compositions have been prepared, they can be place in the appropriate container and labeled for the treatment of Pancreatic Cancer.

While any suitable carrier known may be employed in a pharmaceutical formulation of this invention, the type of carrier will vary depending on the mode of administration and whether a sustained release is desired. Routes of delivery by intra arterial, or other delivery routes which may include oral, intravenous injection, intramuscular injection, subcutaneous injection, inhaled, buccal, intranasal, subdermal, transdermal and intravenous fluid therapy.

For parenteral administration, such as subcutaneous injection or intravenous injection, the carrier can include, for example, any one or more of the following ingredients: adjuvant, water, saline, alcohol, fat, wax, or buffer.

For oral administration, a carrier can comprise carbohydrate or polypeptide fillers, such as sugars, including lactose, sucrose, mannitol, or sorbitol, starch from corn, wheat, rice, potato, or other plants; cellulose, such as methyl cellulose, hydroxpropylmethyl cellulose, or sodium carboxmethylcellulose; gums, including arabic and tragacant; polypeptides such as gelatin and collagen. If desired, disintegrating or solubilizing agents may be added, such as cross linked polyvinyl pyrrolidone, agar, alginic acid, or a salt thereof, such as sodium alginate. If desirable, the drug can be delivered in nanocapsules that would protect against enzymatic or chemical degradation. Such carriers enable the compositions herein to be formulated as tablets, pills, dragees, capsules, liquids, gels, syrups, slurries, suspensions, and the like, for ingestion by the subject. Pharmaceutical preparations for oral use can be obtained through combining active compounds with solid excipient. The resulting mixture can be optionally be ground and processed.

Formulations for topical administration can use a carrier that is a solution, emulsion, and ointment or gel base. The base, for example, may comprise one or more of the following: Petrolatum, lanolin, PEGs, beeswax, mineral oil, diluents such as water and alcohol, and emulsifiers, and stabilizers. Thickening agents may be present in a pharmaceutical composition for topical administration. If intended for transdermal administration, the composition my include an iontophoresis device, transdermal patch or a microneedle transdermal patch.

Biogradable microspheres (e.g. polylactic galactide) may also be employed as carriers for the pharmaceutical compositions of this invention. Suitable biodegradable microspheres are disclosed, for example in U.S. Pat. Nos. 4,897,268 and 5,075,109. Each incorporated by reference herein for all purposes.

Pharmaceutical compositions may also contain diluents such as buffers, antioxidants such as ascorbic acid, low molecule weight (less than about 10 residues) polypeptides, polypeptides, amino acids, carbohydrates, including glucose, sucrose or dextrins, chelating agents such as EDTA, glutathione, and other stabilizers and excipients. Neutral buffered saline, or saline mixed with nonspecific serum albumin are exemplary appropriate diluents. In some cases, the compositions herein are lyophilized using appropriate excipients solution (e.g. sucrose) as diluents.

The compositions herein can be administered in a therapeutically effective dose to treat, mitigate, or prevent Pancreatic Cancer. As used herein, the words “treat”, “treating”, and “treatment” refer to the care provided to improve the condition of a subject afflicted with Pancreatic Cancer. As used herein the words “mitigate” and “mitigating” refer to lessening the extent of or seriousness of Pancreatic Cancer, for example, to lessen, reduce, and/or diminish the effects of or severity of Pancreatic Cancer. As used herein, the words “prevent” and “preventing” refer to stopping or avoiding the effects of multi drug resistance Pancreatic Cancer in a subject. As used herein, the words “limiting the onset” refer to limiting the progression of Pancreatic Cancer during which the subject is exposed to the cell mediated immune response that causes multi drug resistance Pancreatic Cancer.

Pharmaceutically acceptable formulations include compositions wherein active ingredients (e. g., one or more Nedocromil Sodium, analogs of Nedocromil Sodium alone or in combination with one or more of chemotherapy and anti inflammatories are contained in an effective dose to achieve intended purpose. The determination of an effective amount or dosage is well within the capability of those skilled in the art. Typically, an effective dose for Nedocromil Sodium, analogs of Nedocromil Sodium for systemic administration is between 100 mg/kg body weight to 1000 mg//kg body weight or 5% or more concentration of active ingredient per dose. For example, an effective dose for Nedocromil Sodium, analogs of Nedocormil Sodium for intravenous administration is between about 100 mg/kg body weight to about 1000 mg/kg body weight or 5% or more concentration of active ingredient per dose.

For topical, the composition herein may be delivered at dosage up to about 99%, 95%, 90%, 80%, 70%, 60%, 50%, 40%, 30% w/w of the composition. For transdermal, the composition herein may be delivered at dosage up to about 99%, 95%, 90%, 80%, 70%, 60%, 50%, 40%, 30% w/w of the composition.

For example, an effective dose for Nedocromil Sodium, analogs of Nedocromil sodium for intra arterial is between about 10 mg/kg body weight or 5% or more concentration of active ingredient per dose.

As mentioned above, compositions herein may be co-formulated or co-administered with a second therapeutic agent. Examples of therapeutic agents include, but not limited to, analgesics, antipyretic medications (fever reducers), antihistamines, anti inflammatories, antimicrobial agents (e.g., chemotherapy, antiviral agents, and antifungal agents). And combinations thereof.

A therapeutically effective dose refers to the amount of active ingredient which prevents or diminishes the effects of Pancreatic Cancer. The dosage varies depending upon the dosage form employed, sensitivity of subject, and the route of administration. The care giver, in light of factors related to the subject requiring treatment, will determine the exact dosage. Dosage and administration are adjusted to provide sufficient levels of active compound or maintain the desired effect. Factors which can be taken into account include the severity of the disease state, general health, age and weight of subject. Compositions may be administered several times a day for 1 day to 1 week, once a day, twice a day, three times a day, every other day, every 3 to 4 days, every week, once every two weeks, once a month, etc., depending on half-life and clearance rate of the particular formulation and on the risk of or progression of Pancreatic Cancer.

In some embodiments, the subject receives one dose of composition. In other embodiments, the subject receives two or more total dosage of composition. In some embodiments, the subject has chronic Pancreatic Cancer or is prone to Pancreatic Cancer and thus, the subject is administered a composition on a regular basis over an extended period of time.

Normal dosage amounts vary from 100 mg/kg to 1000 mg/kg body weight and/or 5% concentration or more of active ingredient per dose. In some aspects, an effective dose of Nedocromil Sodium, analogs of Nedocromil Sodium is administered by Intravenous injection or intra-arterial. The dosing in this aspect, for example would be 1000 mg or more of effective dose or 5% concentration or more of active ingredient per dose. Guidance as to particular dosages and methods of delivery is provided above, in the literature, and generally available to practitioners in the art.

For topical administration or transdermal including Intra Arterial lower dosage may be required. For repeated administrations over several days or longer depending on the condition, the treatment is sustained until Pancreatic Cancer is avoided or suppressed in severity. However, other dosage regimens may be useful.

It is anticipated that different formulations will be effective for different treatment compositions i.e. an effective dose of Nedocromil Sodium, analogs of Nedocromil Sodium alone or in combination with chemotherapy, anti inflammatories, antihistamines, and/or analgesics.

Nedocromil Sodium, analog of Nedocromil Sodium compositions may be administered in the form of a solid, liquid, spray, or gas (aerosol). For example, for oral administration as a pharmaceutical formulation, the composition can be delivered as syrup, lozenger, pill, gel, capsule, etc. For subdermal or subcutaneous delivery, it can be delivered in a liquid formulation. For topical and/or transdermal administration, the pharmaceutical composition can be delivered as a gel, cream patch or microneedle transdermal patch. For delivery to the lungs or brain, the composition can be in a liquid spray, spray of fine solid particles, or gas.

Thus, provided herein is a pharmaceutical formulation comprising, consisting of, or consisting essentially of Nedocromil Sodium, analogs of Nedocromil Sodium compositions and a pharmaceutical excipient.

Any of the excipients described herein or any other ones known in the art can be used according to the present invention.

The pharmaceutical composition is formulated so as to allow the active ingredient(s) contained therein to be bioavailable upon administration of the composition to a subject. Compositions that will be administered to the subject take the form of one or more dosage units, where for example, an intra-arterial administration may be a single dosage unit and a container of one or more compounds of the invention in aerosol form may hold plurality of dosage units.

For oral administration, an excipient and/or binder may be present. Examples are sucrose, kaolin, glycerin, starch dextrins, sodium alginate, carboxymethylcellulose and ethyl cellulose. Coloring and or flavoring agents may be present. A coating shell may be employed.

The composition may be in the form of a liquid, e.g., an elixir, syrup, solution, emulsion, or suspension. The liquid may be for oral administration or for delivery by injection, as two examples. When intended for oral administration, compositions can contain in addition to the compositions herein one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent can be included.

Injectable formulations of the compositions herein are preferably sterile. Means for achieving sterility are well known in the art.

For delivery to the dermis and/or epithelium, dermal patches, microneedle dermal patches and delivery systems utilizing active or passive transdermal delivery carriers can be prepared using well known methods and materials, including, for example, microporous membranes, silicon polymers and diffusion matrixes. Such materials and methods are described, for example in Remington's Pharmaceutical Sciences, supra.

The compositions (including formulations) herein can be administered systemically or locally to a subject by any means known in the art. For example, to a subject, the compositions herein can be administered parenterally by intra arterial which also includes subcutaneously, intravenously, intramuscularly, intrasternally, intracavernously, intrathecally, and intraurethrally), intracranially, intraorbitally, intracapsularly, intraspinally, intracistemally, intrapulmonarily (via inhalation) orally, intravenously, intra-arterially, intramedullary, intrathecally, intraventricularly, transdermally (which includes microneedle patch), subcutaneously, intraperitoneally, intranasally, enteraslly, vaginally, sublingually, intratumorally or rectally. In some embodiments, the composition/formulations herein are administered using inser(s), bead(s), time released formulations, patch (es, microneedle) or fast or slow release formulation(s).

The compositions/formulations herein are administered in an effective dose. It will be evident to those skilled in the art that the number, frequency, and duration of administration will be dependent upon the response of the subject.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

All references cited herein are incorporated by reference in their entirety and to the same extent as if each individual publication or patent application was specifically and individually indicated to be incorporated by reference.

The following examples are provided for illustrative purposes only and are not intended to limit the scope of the invention. In examples “The compound” or “compound” refers to composition Nedocromil Sodium.

### Examples

Summary—This year in the United States, an estimated 42,470 patients will be diagnosed with PDAC and 35,240 will die of their disease, making PDAC the most lethal of all solid tumors. This horrible prognosis is largely due to the fact that PDAC is highly resistant to systemic therapies. Only around 20% of patients are candidates for surgery, which is the most effective known therapy for PDAC. The clinical standard of care for pancreatic ductal adenocarcinoma (PDAC) is treatment with gemcitabine (Gem). Gem has minimal survival benefits and is primarily used for its palliative properties. Several mechanisms exist for resistance to this drug However, we have found that inhibition of the receptor GPCR 35 (Cell Immune receptor) sensitize even highly resistant cells and improves responses to cytotoxic chemotherapies both in vitro and in vivo in mouse models.

Summary-G protein-coupled receptor 35 also known as GPR35 is a cell immune receptor which in humans is encoded by the GPR35 gene. Heightened expression of GPR35 is found in immune and gastrointestinal tissues, including the pancreas. High levels of human GPR35 expression has been observed in bacteria, macrophages, mast cells, monocytes, T cells, natural killer cells, neutrophils, eosinophils, and dendritic).

Summary—The Compound was shown to be a potent full agonist of GPR 35 by implementing PathHunter human GPR35a-β-arrestin-2 interaction assays were performed using a CHO-K1 cell line stably expressing human GPR35 and β-arrestin (EC₅₀=2.98±1.27 μM).

Summary—Whole-cell patch-clamp recordings were used to test the effects of The Compound on ion channels. The compound was shown to block ion channels present in the intestinal cell line HT29 (ICs0 19 ixmol/L) and acted in a dose-dependent manner.

Summary—Persistent and Irreversible Blockade of ca2+ Pools by compound in cultured cells-Previous experiments characterizing the blockade of Ca2+-pumping activity by The Compound in permeabilized cells had been undertaken by observing the effects of the compound added directly within Ca2+ transport assays. An interesting observation has arisen from experiments in which compound was added to cells growing in culture. In these studies, DDT, MF-Z cells were treated with 3 p˜compound shortly after passaging for a brief (30-min) period. Following this treatment, the culture medium was replaced with standard compound-free medium, and the cells remained in culture for a further 7 days, during which time there were three further medium changes with the same compound-free medium. When such treated cells were harvested and permeabilized under standard conditions, it was clear that the Ca2+-pumping activity responsible for Ca2+ uptake into the InsP3- and GTP-sensitive Ca2+ pools was still completely inactivated.

Summary—Mast cell infiltration significantly increased in pancreatic cancer compared to normal pancreatic tissue [11.4±6.7 vs. 2.0±1.4(p<0.001)]. Increased infiltrating mast cells correlated with higher grade tumors (p<0.0001) and worse survival. Patients with pancreatic cancer had elevated serum tryptase activity (p<0.05). In vitro, AsPC1 and PANC-1 cells induced mast cell migration. Mast Tumor infiltrating mast cells are associated with worse prognosis in pancreatic cancer. In vitro, the interaction between mast cells and pancreatic cancer cells promote tumor growth and invasion.

Summary—Research Trial of Excindogen at MD Anderson Cancer Center

Blocking mast cell degranulation results in therapeutic responses to PDAC in vivo

To determine whether targeting mast cell function has a therapeutic effect in PDAC, we injected tumor-bearing C57BL/6 mice Nedocromil sodium, a mast cell stabilizer. The growth of surgically implanted K-ras^{G12D}p53^{-/-} tumors was significantly suppressed by Nedocromil treatment; P<0.001 treated mice vs. saline control at day 42 after treatment). Tumor measurement was performed at the end time point (day 50) after treatment. The mean tumor sizes in the saline-treated and Nedocromil treated groups were 31.3 and 5.2 mm³, respectively. A P value of 0.017 was determined using Student's t-test. Similarly, mast cell degranulation in the tumor microenvironment was inhibited in the nedocromil sodium-treated group compared with in the saline-treated control group. The growth of Panc-02 tumor cells was also significantly suppressed by nedocromil treatment; P<0.001 treated mice vs. saline control at day 21 after treatment). These data further confirm the critical role of mast cells in PDAC progression and suggest that therapies that target mast cell degranulation maybe a useful adjuvant treatment for PDAC.

Summary—A female dog was given the diagnosis of a cancerous tumor (without confirmatory histology due to lack of ability to aspirate the tumor due to the hardness of the mass) in June of 2020. At the time of diagnosis the tumor located on his right shoulder was measured at 2 inches by 2 inches with a depth of 0.5 inches. Other than supportive care (ketogenic diet to delay tumor progression, and gabapentin for neuropathic pain) no treatment was provided by the owners, as the cost and lack of clear benefit to conventional therapy (biopsy, chemotherapy, and eventual resection) was not deemed to be appropriate for this dog. By December of 2020, the tumor was measured at 5.5 inches by 4.5 inches with a depth of 1.5 inches

On December 23^{rd}, 2020 the first application of Nedocromil sodium compound was administered to the dog via the topical route. The dog's tumor area was shaved to improve the absorption of the compound into the skin. On Jul. 16, 2021 (tumor size of 1.5×1×0.5 inches), a second application of Nedocromil Sodium was applied using the same dosing strategy in attempt to eradicate the tumor entirely. Following a similar timeline as before, roughly 6 weeks after application the tumor was noticeably smaller, and by the 8 week mark, no tumor was able to be felt by the dog's owner.

Summary—Transit Metastasis Melanoma Case Study

70 year old man with stage 4 transit metastatic melanoma with 30 melanomas on the body. Using chemotherapy infusions every 3 weeks since 2019. For 2 weeks used topical Nedocromil sodium with one dose per tumor (30) per week. After one treatment of Nedocromil Sodium the tumors have flattened. This phenomenon is the process of the tumors resolving. In modern medicine the goal is to shrink a tumor, but the tumor never resolves. Excindogen has the capability of dissolving the tumor. No other chemotherapy drug has this capability. You can see from the photos that in one week changes have occurred where the prescribed T-Vec treatment takes up to 5 months to see a clinical effect. AFTER 2nd TREATMENT 17 OF THE 30 TUMORS DISSOLVED. The patient was treated with Nedocromil Sodium on all 30 tumors in one sitting. Absolutely no side effects.

What is claimed is:

1. A method for the treatment of Pancreatic cancer, methods and compositions are equally applicable to any type of Cancer herein can include:
Bladder Cancer
Breast Cancer
Colon and Rectal Cancer
Endometrial Cancer
Kidney Cancer
Leukemia
Liver Cancer
Lung Cancer
Melanoma
Non-Hodgkin Lymphoma
Prostate Cancer
Thyroid Cancer
the method comprising administrating Nedocromil Sodium or analog of Nedocromil Sodium to a subject in need thereof, where in Nedocromil sodium or analog of Nedocromil Sodium treats Pancreatic Cancer in the subject or from list above.

2. The method of claim 1, wherein the subject is human or animal.

3. The method of claim 1, where Nedocromil Sodium or analog of Nedocromil sodium is administered one time, two times, daily, every other day, every three days, every four days, every five days, every six days, twice weekly, weekly, every other week. Twice a month, monthly, or every other month.

4. The method of claim 1, wherein the subject suffers from recurring chronic Pancreatic Cancer and wherein Nedocromil Sodium or analog of Nedocromil Sodium is administered on a daily basis.

5. The method of claim 1, wherein composition and formulation is selected from Nedocromil Sodium or analog of Nedocromil Sodium.

6. The method of claim 5, wherein Nedocromil Sodium.

7. The method of claim 6, wherein Nedocromil Sodium is administered by microneedle transdermal patch or Intra arterial.

8. The method of claim 1, the method further comprising administering to the subject a medicament selected form the group consisting of chemotherapy, anti-inflammatories and analgesic.

9. The method of claim 1, where in Nedocromil Sodium or analog of Nedocromil Sodium is administered in a dose of 500/kg to at least 1000 mg/kg subject body weight and/or 5% concentration or more of active ingredient per dose.

10. A method of preventing Pancreatic Cancer, the method comprising identifying a subject at risk of Pancreatic Cancer and administering Nedocromil Sodium to the subject in an amount effective for preventing Pancreatic Cancer.

11. The method of claim 10, where Nedocromil Sodium is administered intra arterial.

12. The method of claim 10, wherein Nedocromil Sodium is administered daily.

13. The method of claim 10, wherein Nedocromil Sodium is administered daily until the risk of Pancreatic Cancer is diminished.

14. The method of claim 10, the method further comprising administering to the subject a medicament selected from the group consisting of chemotherapy, anti-inflammatories, and analgesic.

15. A method of limiting onset of Pancreatic Cancer, the method comprising identifying a subject at risk of Pancreatic Cancer and administering Nedocromil Sodium to the subject in an amount effective for limiting onset of Pancreatic Cancer.

16. The method in claim 15, wherein Nedocromil Sodium is administered intra arterial.

17. The method of claim 15, wherein Nedocromil Sodium is administered daily.

18. The method of claim 15, wherein Nedocromil Sodium is administered daily until the risk of Pancreatic Cancer is diminished.

19. The method of claim 15, the method further comprising administering to the subject a medicament selected from the group consisting of chemotherapy, anti-inflammatories, and analgesic.

20. A method for treating chronic Pancreatic Cancer, the method comprising administering Nedocromil Sodium to a subject in need thereof.
